(19) United States
(12) Patent Application Publication
(10) Pub. No.: US 2016/0271063 A1
(43) Pub. Date: Sep. 22, 2016

(54) STERILE S-ADENOSYL METHIONINE WITH A HIGH CONTENT OF ACTIVE ISOMER FOR INJECTABLE SOLUTIONS, AND PROCEDURE FOR OBTAINING IT

(71) Applicant: GNOSIS S.P.A., Milano (IT)

(72) Inventors: Marco Berna, Desio (IT); Auro Tagliani, Desio (IT); Daniele Gregori, S. Antonino (CH)

(73) Assignee: Gnosis S.p.A., Milano (IT)

(21) Appl. No.: 14/778,032

(22) PCT Filed: Mar. 19, 2014
(86) PCT No.: PCT/IB2014/059966

§ 371 Date: Sep. 17, 2015

(30) Foreign Application Priority Data
Mar. 20, 2013 (IT) MI2013A000426

(51) Int. Cl. A61K 9/16; A61K 9/00; A61K 31/7076

(52) U.S. Cl. CPC A61K 9/1682; A61K 31/7076; A61K 9/0019

(57) ABSTRACT

S-adenosyl methionine, and the salts and complexes thereof, in the form of a spray-dried sterile powder which has a pharmacologically active enantiomer content exceeding 70% and a water residue below 2.5% by weight.

### SUMMARY

The present invention relates to a sterile S-adenosyl-L-methionine (SAMe) in the solid state in amorphous form, and the process for obtaining it.

The SAMe of the present invention is characterised by a higher degree of purity and stability and a higher content of active isomer, (S,S)-S-adenosyl-L-methionine, than the product obtained by known techniques.

The SAMe of the present invention preferably takes the form of sulphonic salts, preferably sulphate, 1,4-butanedisulphonate and para-toluenesulphonate.

### BACKGROUND

S-Adenosyl-L-methionine (SAMe) is present in all living organisms, in which it performs the role of the most important methylating agent in the cell metabolism.

In view of its ubiquitous role, a deficiency of this important vitamin in the human body contributes to the onset of numerous disorders. For example, a SAMe deficiency in the body is associated with the development of osteoarthritis, cirrhosis of the liver, cystic fibrosis, some states of depression, and disorders related to aging such as Alzheimer's and Parkinson's disease. Moreover, low levels of SAMe are associated with the development of cardiovascular disorders.

S-Adenosyl-L-methionine (SAMe) is highly unstable at temperatures exceeding 0° C. SAMe breaks down rapidly at ambient temperature, giving rise to S-adenosylhomocysteine (SAH), homoserine, methylthioadenosine (MTA), S-5 ‘-adenosyl-(5’)-3-methylpropylamine or decarboxylated SAMe (dcSAMe) and adenine. Low pH values and humidity percentages are among the main factors involved in preserving SAMe against chemical degradation.

S-Adenosyl-L-methionine exists in two diastereoisomeric forms: (S,S)-S-adenosyl-L-methionine and (R,S)-S-adenosyl-L-methionine.

The natural S-adenosyl-L-methionine produced by the body is biosynthesised using cysteine as substrate to give a single diastereoisomer: (S,S)-S-adenosyl-L-methionine, which is the biologically active isomer.

The other diastereoisomer [(R,S)-SAMe] is not only inactive in the numerous physiological functions of SAMe, but is considered by some researchers to possess an opposite, and therefore potentially harmful activity (US2005/0272687, Borchardt and Wu, J. Med. Chem.; 19 (9), 1099, 1976).

The S-adenosyl-L-methionine obtained by synthesis consists of a 50%-50% diastereoisomeric mixture of (S,S)-S-adenosyl-L-methionine and (R,S)-S-adenosyl-L-methionine. The natural SAMe extracted as (S,S)-SAMe also tends to racemise, adjusting the relative ratio between the two diastereoisomers towards a 50%-50% balance over time. The adverse influence of racemisation of SAMe samples on the efficacy of an osteoarthritis treatment was described by Najm et al. in BMC Musculoskelet. Disord.; 5 (1), 6, 2004. Temperature control during the SAMe purification process is a key factor in limiting racemisation of SAMe (US2005/0272687), as temperature and pH are the main factors able to accelerate or slow the racemisation process of the molecule.

Numerous attempts have been made to obtain stable salts of S-adenosyl-L-methionine.

One way of limiting racemisation is to bond the SAMe with counterions or complexing agents. Numerous counterions are known for obtaining SAMe salts with increased stability, but they only limit the stability problem, without solving it completely (US2005/0272687, U.S. Pat. No. 6,649,753).

The most stable products are SAMe salts with medium and strong acids, especially organic and inorganic carboxylic and sulphonic acids. The most widely used are 1,4-butanedisulphonic, p-toluenesulphonic and sulphuric acid; they are also applied for injectable formulations of SAMe. In view of the very low pH that would be obtained, in order to limit physiological problems (pain and tissue damage), these salts can be dissolved with a buffered solution, e.g. with lysine.

SAMe is mainly administered orally: the vast majority of SAMe-based preparations in the form of diet supplements involve oral administration of tablets or capsules wherein the molecule is stabilised or protected in various ways to preserve its chemical integrity and protect the gastric mucosa against the high acidity of the product.

However, injectable formulations of SAMe are important when the molecule needs to reach areas other than the liver, such as the central nervous system. Intramuscular or intravenous administration routes guarantee that the product will reach the target organs in active form, without previously passing through the liver. Sterile solutions of SAMe can be freeze-dried, to prevent the breakdown of the product, either as bulk product or after vial filling; the procedure commonly used to prepare the finished pharmaceutical form is freeze-drying after vial filling, in a sterile environment. A vial of solvent containing a buffer, such as a suitable quantity of lysine, is added to the powders obtained in this way to counteract the high acidity of the SAMe salts. Direct freeze-drying in vials is the option most commonly used for the manufacture of injectable medicaments based on SAMe, although it is the most expensive option and involves a slight degradation of the product; for example a certain degree of racemisation is observed, which reduces the percentage of active enantiomer to under 70%.

There is consequently a need for a form of SAMe suitable for parenteral administration which guarantees improved characteristics of sterility, stability and purity.

### BRIEF DESCRIPTION OF THE DRAWINGS

FIG. 1 shows the particle-size distribution of the spray-dried product of the present invention.

FIG. 2 shows the particle-size distribution of the freeze-dried product.

FIG. 3 is a microscope photograph of the spray-dried product of the present invention.

FIG. 4 is a microscope photograph of the freeze-dried product.

FIG. 5 shows the X-ray diffraction spectrum of the spray-dried product, recorded at al and a 2 copper radiation.

### DETAILED DESCRIPTION

It has now been found that S-adenosyl methionine can be obtained in stable, sterile form with a high content of pharmacologically active enantiomer by means of a spray-drying process.

The object of the invention is therefore S-adenosyl methionine, and the salts and complexes thereof, in the form of a spray-dried sterile powder which has a pharmacologically active enantiomer content exceeding 70% and a water residue below 2.5% by weight.

The S-adenosyl methionine of the invention is amorphous, as demonstrated by the X-ray diffraction spectrum.

The invention also relates to the process for the preparation of said form of S-adenosyl methionine.

The S-adenosyl methionine of the invention preferably has a water content below 2% by weight and an active enantiomer content exceeding 75% of the total SAMe. The enantiomer content is determined by the percentage of the area of active isomer to the sum of the area of both isomers in the HPLC analysis.

The S-adenosyl methionine of the invention is characterised by a spheroid particle shape and particle sizes of less than 100 microns, in particular between 8 and 50 microns, and a surface area of less than 0.5 m2/g. By suitably regulating the operating conditions, a particle size of less than 10 microns can also be obtained, which is particularly suitable for use in preparations for inhalation, as described in EP1238665.

The S-adenosyl methionine of the invention is prepared by spray-drying a solution or suspension of S-adenosyl methionine or a salt thereof, preferably the salt of 1,4-butanedisulphonic acid (hereinafter called “SAMe SD4”) or the mixed sulphate/p-toluenesulphonate salt (hereinafter called “SAMe Pates”).

Said salts are highly hygroscopic solids, characterised by high chemical purity and high solubility in water, which can exceed 250 g/l of SAMe ion in solution.

The solutions of SAMe SD4 and SAMe Pates are stable, unless microbial contamination is present, provided that they are stored at a cold temperature, as they break down rapidly at ambient temperature.

The starting SAMe can be obtained from biotransformation processes, for example from yeast, according to the procedures described in IT8420938.

The spray-drying process consists of spraying a solution or suspension of the product in a solvent, usually water, in an environment at a temperature that allows rapid evaporation of the solvent, thus leaving the dry product in the form of an amorphous powder. The process can be very rapid, to prevent breakdown of the product, and is generally conducted in a hot air flow (or nitrogen if flammable solvents are present). Conventional equipment available on the market is used, such as the Buchi Mini Spray Dryer B-290, which is suitable for laboratory tests, or the Mobile Minor manufactured by GEA Niro, which is suitable for tests on a pilot scale or small production runs. Other spray-drying units, which are similar to those described above but suitable for use in the manufacture of sterile powders, are available on the market, and operate on the same principles as the instruments cited. They are fed with a sterile drying gas, such as air or nitrogen, filtered under sterile conditions through HEPA filters, and discharge the product into suitable closed vessels. The spray dryer, which is wholly or partly maintained in an aseptic controlled-class environment, is fed with a sterile solution of the product. The unit can be equipped with nozzles for cleaning (clearing in place, CIP) and sterilisation (sterilisation in place, SIP), for example by steam.

The SAMe solution fed in is pre-filtered through a sterilising filter (for example an 0.2 micron polymer or ceramic filter) and conveyed to the spray dryer through pre-sterilised metal or polymer pipes. Other water-soluble substances can be added to the SAMe solution, such as buffers, diluents or other co-formulants, and can be sterilised by filtration through suitable sterilising filters, for example through 0.2 micron polymer filters.

The temperature of the air entering the drying chamber ranges between 130 and 190° C., preferably between 135 and 160° C. The temperature of the outgoing air is regulated in the interval between 105 and 75° C., preferably 97 to 85° C., by suitably varying the input flow rate of the solution.

The conditions specified produce SAMe in sterile solid form and limit the breakdown of the product, including racemisation, giving rise to a product of quality equal to or greater than that of the freeze-dried product.

The product obtained also combines the best characteristics of spherical shape, particle-size distribution and other physical properties, thus providing the product with good flowability and simultaneously a good degree of packing. This allows the use of existing vial-filling machines, which operate in an aseptic environment, according to vacuum and pressure mechanisms. The product is aspirated into these machines from the loading hopper in a batching chamber of suitable volume, fitted with a suitable filter, and then expelled into the vial by applying a slight pressure.

The flowability and particle-size characteristics of the powder are of crucial importance, in the case of medicaments, to guarantee an accurate dose: a powder which is not very flowable and/or has an irregular shape may not completely fill the chamber, leaving small empty spaces that make the dose imprecise, whereas a powder that is too fine tends to elude the filters, thus increasing wastage of the active ingredient, with a consequent increase in the cost of the medicament. In the particular case of SAMe and its salts, the difficulty is increased by the strongly hygroscopic characteristics of the product: the powder is fairly flowable if perfectly dry, but tends to absorb humidity from the environment, and becomes sticky as the degree of humidity increases. Even if the filling chamber is not completely occluded, variations are observed in the physical properties of the powder that make the dose imprecise, with the risk that vials with different doses of medicament will be found in the same batch. For these reasons, it is of crucial importance to have a perfectly dry powder, with spherical particles having a sufficiently homogenous particle-size distribution; when applied to vial-filling machines for powders, a high level of dose homogeneity can be obtained, with variations lower than +5% from beginning to end of the batch, even at doses below 1 gram.

Vial-filling with powders is usually performed with machines having a very high output, which dispense accurate doses and handle a large number of vials, such as thousands of vials per hour. To work with this productivity and with the necessary dose precision, it is essential for the batched powder to have precise, constant flowability and particle-size characteristics: any variation leads to a large number of rejects per manufacturing batch due to an imprecise dose, or requires the output of the machine to be reduced.

An example of such vial-filling machines is the MF400 made by IMA Life Science, which can fill up to 400 vials a minute with adequate doses of SAMe-based drugs. In the specific case of SAMe, as it is a very hygroscopic powder, it is essential to prevent the product from absorbing humidity from the environment; particular precautions are therefore required, for example the use of a sterile, dry environment, such as dehumidified air filtered through HEPA filters. In the case of SAMe, it is all the more important for the powder to remain perfectly dry, because in these conditions it presents good flowability and greater chemical stability.

The invention is described in greater detail in the examples below.

### Example 1

S-Adenosyl methionine is produced by biotransformation with yeast, as described in patent IT8420938. At the end of the purification process a solution of SAMe salified with 1,4 butanedisulphonic acid is obtained, which is concentrated by nanofiltration and/or evaporation under vacuum to a concentration of about 125-250 g/l of SAMe.

A solution of SAMe 1,4-butanedisulphonate at the concentration of about 150 g/l of SAMe, amounting to about 31% of the total solids, is fed by a peristaltic pump into a GEA Niro Mobile Minor drier as described above, pre-heated to the desired temperature. The apparatus is fitted with a dual-fluid nozzle injector and supplied with hot air as drying gas and cold air for pressurisation and cooling of the injector; the air entering the drying chamber is heated by heating elements to the temperature of 150° C., hereinafter indicated as the “input temperature” (TIN). The supply of SAMe solution to the system is adjusted by manually regulating the flow rate of the peristaltic pump to obtain the desired temperature value for the outgoing air (TOUT), amounting to 95-96° C. The SAMe 1,4-butanedisulphonate powder is separated by the cyclone and collected in a glass jar, while the damp air is expelled.

The product thus obtained has an assay value as SAMe of 48.6%, residual humidity 1.50%, stereoisomeric ratio 73.77%, particle size D50=10 microns.

### Example 2

The process is performed as described in Example 1, setting the input and output temperatures as described in the table below. The product obtained has the characteristics reported in Table 1.

TABLE 1
Test in Mobile Minor unit

| Test | 2 | 3 | 4 | 5 | 6 | 7 | 8 | 9 | 10 | 11 |
|---|---|---|---|---|---|---|---|---|---|---|
| IN temperature | 137 | 137 | 160 | 130 | 177 | 160 | 150 | 147 | 182 | 157 |
| OUT temperature | 90 | 90 | 102 | 85 | 95 | 84 | 97 | 75 | 85 | 95 |
| Particle size D₅₀μ | 9 | 9 | 8 | 9 | 8 | 10 | 9 | 10 | 10 | 10 |
| Humidity % | 2.07 | 1.63 | 1.63 | 1.88 | 2.07 | 2.65 | 1.54 | 3.51 | 2.49 | 1.61 |
| S,S Stereoisomer % | 75.80 | 75.65 | 72.60 | 76.63 | 71.16 | 75.95 | 73.15 | 77.56 | 74.56 | 73.1 |
| SAMe % | 48.53 | 48.54 | 48.46 | 48.33 | 48.44 | 48.15 | 48.66 | 47.89 | 48.03 | 48.43 |

### Example 3

The process is performed as described in Example 1, introducing some modifications to the drying unit: a HEPA sterilising filter is introduced onto the air supply line, and a sterilising filter cartridge is placed on the delivery line of the peristaltic pump, between the pump and the injector of the spray dryer.

The process is performed under the same conditions as described in example 2, after pre-sterilising the dryer, the tank, the peristaltic pump, the feed pipes of the SAMe solution, and the powder collection vessel. Each part of the unit is sterilised with steam or chemical disinfectants, depending on the compatibility of the materials, and the SAMe solution is sterilised by filtration and stored cold (+4° C.).

A solid product is obtained which has the same chemical characteristics as the products reported in Table 1, but characterised by an enantiomeric excess exceeding 80% of S,S isomer, a total microbial count of less than 10 CFU/g, and an endotoxin level of less than 0.118 U/g.

### Example 4

The same unit is used as described in example 3, with the addition of a heat exchanger supplied with a cooled mixture and a Munter chemical dryer on the drying gas line, after the powder separation cyclone. The exhausted drying gas, after being dehumidified, is returned to the system, which is therefore a closed cycle.

The process is performed as described in Example 3, using nitrogen as drying gas and operating under the same conditions as described in Example 2.

A solid product is obtained which has the same chemical characteristics as the products reported in Table 1, but characterised by an enantiomeric excess exceeding 80% of S,S isomer, a total microbial count of less than 10 CFU/g, and an endotoxin level of less than 0.118 U/g.

### Example 5

The same unit is used as described in example 4, employing a solution of SAMe 1,4-butanedisulphonate at about 250 g/l.

The temperature of the input gas is set as indicated in Table 1, and the flow rate of the SAMe feed solution is then suitably regulated to comply with the corresponding output temperature, as indicated in Table 1.

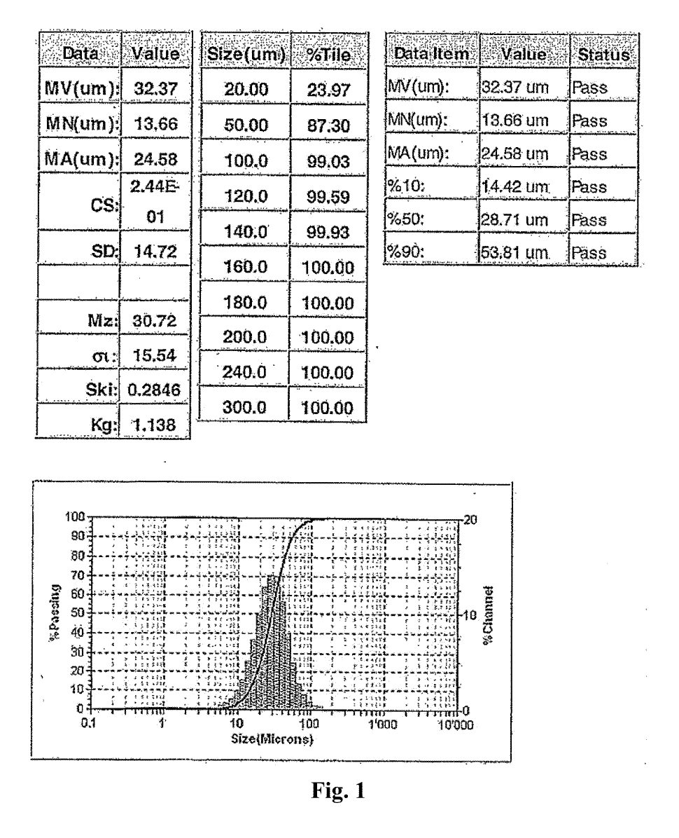

A product is obtained which has the chemical characteristics described in Table 2 and the microbiological characteristics described in Example 4; moreover, the solid particles obtained have the appearance of spheres as shown in FIG. 3, with a homogenous particle size distribution as shown in FIG. 1.

TABLE 2
Chemical analysis of the product described in the
text compared with the medicament on the market

| Test | SAMYR | Example 5 |
|---|---|---|
| HPLC identification | Complies | Complies |
| SAMe ion (% odb) | 48.5% | 51.6% |
| SAO (% area) | 0.52% | 0.14% |
| ADN (% area) | 0.54% | 0.26% |
| MTA (% area) | 0.88% | 0.28% |
| Humidity | 0.70% | 1.20% |
| SS isomer (%) | 63.9% | 73.1% |
| HPLC USP - purity | 97.34% | 98.56% |
| (% area) | | |
| HPLC USP - max impurity | 1.32% | 0.69% |
| (% area) | | |
| BET surface area (m2/g) | 0.6534 | 0.3460 |
| Microscope | Irregular, | Regular, |
| | agglomerated particles | spherical particles |
| Laser particle-size | 10% = 11.68 micron | 10% = 14.4 micron |
| distribution analysis | 50% = 70.09 micron | 50% = 28.7 micron |
| | 90% = 489.5 micron | 90% = 53.8 micron |

### Example 6

The same unit is used as described in Example 3, employing a solution of SAMe sulphate and p-toluenesulphonate at a concentration ranging from 100 to 250 g/l, and operating under the conditions described in Example 4.

A SAMe Pates powder with the following characteristics is obtained:

Assay value as SAMe exceeding 49.5%, enantiomeric ratio exceeding 80% of the S,S isomer, total impurities lower than 3.5%, particle size less than 50 microns, bulk density less than 0.7.

### Example 7

A solution of about 125 g/l of SAMe 1,4-butanedisulphonate is divided between glass vessels and freeze-dried using an MF680-MK2 Edwards freeze-drying chamber programmed according to the following operating cycle.

Freezing of the solution by cooling to −45° C. for at least 3 hours.

Primary drying under vacuum at 0.04 mBar with a maximum temperature of −10° C. for 30 hours, then under vacuum at 0.010 mBar at temperatures of −10° C. for 35 hours until stable pressure is reached from a minimum of 10 hours at 0.010 mBar.

Secondary drying at +20° C. under vacuum at 0.010 mBar for 3 hours, then at +45° C. under vacuum at 0.010 mBar for at least 6 hours.

Cooling at +20−25° C. under vacuum, followed by introduction of nitrogen into the freeze dryer.

The powder obtained has an assay value as SAMe of 48%, and an enantiomeric ratio of 70% S,S isomer.

### Example 8

The powder obtained by spray drying as described in Example 6 is placed in an IMA Life Microfill 400 filling machine, placed under an aseptic isolator and pre-sterilised.

The aseptic isolator is supplied with air dehumidified with a Munter drier and filtered under sterile conditions with HEPA filters, while the machine is supplied with individually pre-weighed glass vials, with aluminium caps and crimps.

When the filling machine is started, the vials are filled with 800 mg of powder per vial at the maximum operating speed of the machine, obtaining a powder dose variability of less than 3%.

### Example 9

The powder obtained by freeze-drying as described in Example 7 was placed in the Microfill 400 machine, operating as described in example 8 for vial-filling with a dose of 800 mg.

A variability in powder dose exceeding 5% is obtained at the maximum operating speed of the machine.

### Example 10

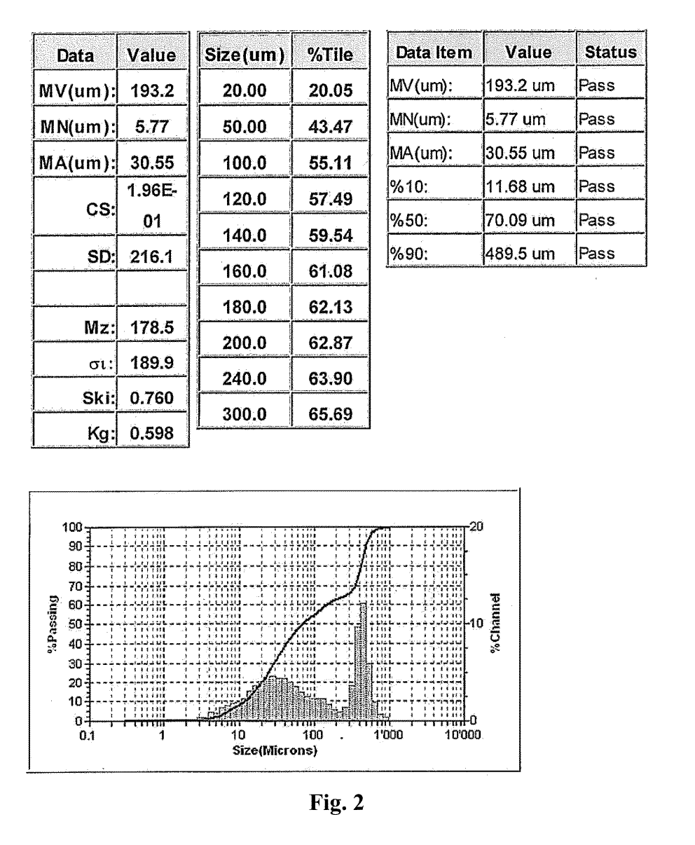

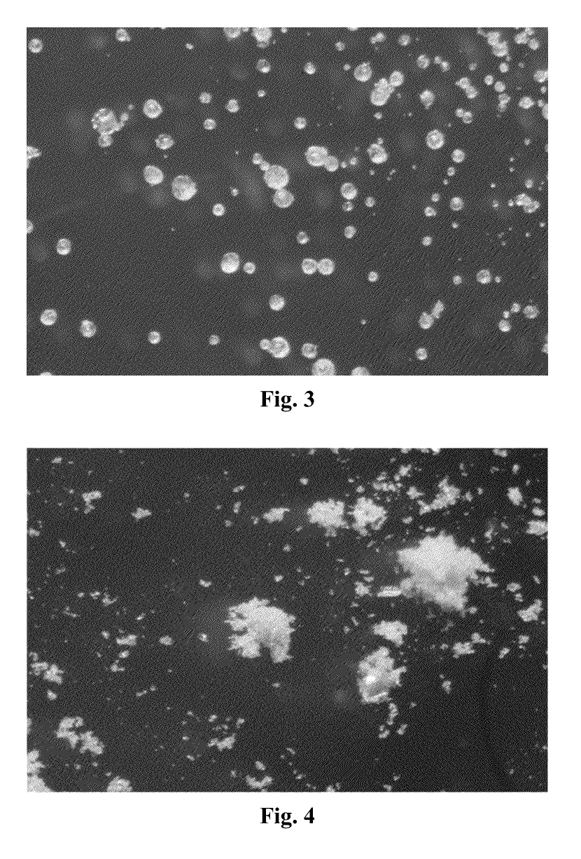

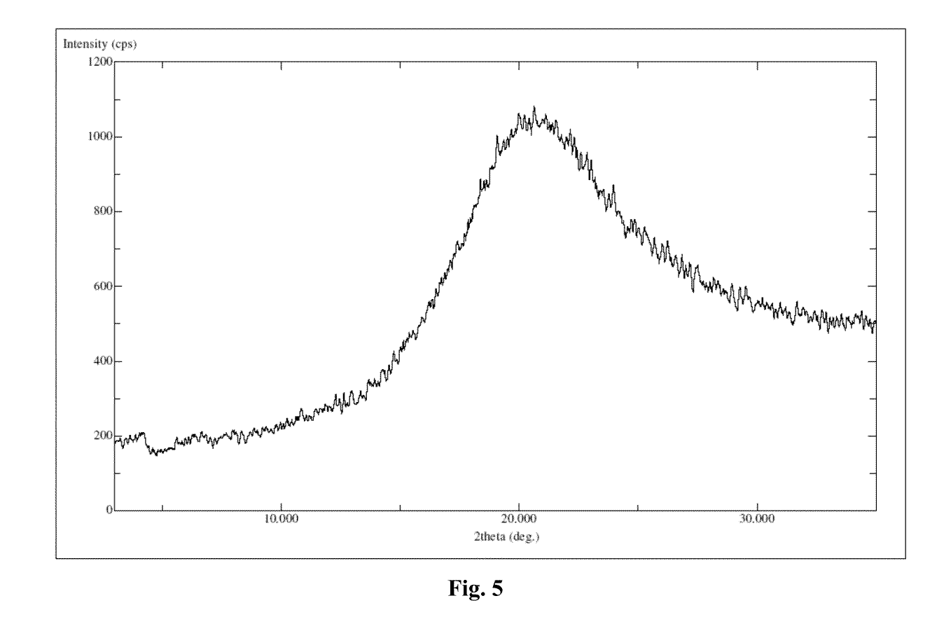

Samyr, a commercial product based on SAMe 1,4-butanedisulphonate, which is on sale in pharmacies, was subjected to the same analyses as described in Example 5. The product has the chemical characteristics described in Table 2, and the solid particles obtained have the irregular appearance shown in FIG. 4, with a heterogeneous particle-size distribution as shown in FIG. 2.

1. S-Adenosyl methionine, and salts and complexes thereof, in the form of a spray-dried sterile powder having:
a pharmacologically active enantiomer content higher than 70%;
a water residue lower than 2.5% by weight;
a spheroid shape of the particles and a particle size distribution ranging from 8 to 50 micron; and
a surface area lower than 0.5 m²/g.

2. S-Adenosyl methionine according to claim 1, having a water content lower than 2% by weight.

3. S-Adenosyl methionine according to claim 1, having an active enantiomer content higher than 75%.

4. S-Adenosyl methionine according to claim 1, in the form of 1,4-butanedisulphonic acid salt.

5. S-Adenosyl methionine according to claim 1, in the form of mixed sulphate/p-toluenesulphonate salt.

6. A process for the preparation of the S-adenosyl methionine according to claim 1, which comprises feeding a solution of S-adenosyl methionine or of a salt thereof in water, previously sterilised by filtration, to a spray dryer under sterile conditions with an inlet air temperature in the drying chamber ranging from 130 to 190° C., and an outlet air temperature adjusted in the range from 105 to 75° C.

7. The process according to claim 6, characterised by the use of a unit which can be sterilised in situ with a dual fluid nozzle injector.

8. An injectable sterile formulation containing S-adenosyl methionine or salts and complexes thereof according to claim 1, to be reconstituted before use with a sterile solvent contained in a separate vial.

9. The injectable sterile formulation according to claim 8, comprising a vial containing a SAMe salt and a pH buffering salt to be reconstituted before use with water for injections free from buffering agents.

10. S-Adenosyl methionine according to claim 1, having an active enantiomer content higher than 80%.
